(19) United States
(12) Patent Application Publication
(10) Pub. No.: US 2016/0346241 A1
(43) Pub. Date: Dec. 1, 2016

(54) ORAL PHARMACEUTICAL COMPOSITION OF ISOTRETINOIN

(71) Applicant: Sun Pharmaceutical Industries Limited, Mumbai (IN)

(72) Inventors: Pankaj Prabhakar AMRUTKAR, Ahmednagar (IN); Sumit MADAN, New Delhi (IN); Ravi KOCHHAR, Gurgaon (IN); Subodh DESHMUKH, Gurgaon (IN)

(21) Appl. No.: 14/958,532

(22) Filed: Dec. 3, 2015

Related U.S. Application Data
(63) Continuation of application No. PCT/IB2015/054090, filed on May 29, 2015.

(51) Int. Cl. A61K 31/203; B29C 43/00; A61K 9/48; A61K 9/00; A61J 3/07

(52) U.S. Cl. CPC A61K 31/203; A61K 9/0053; A61J 3/07; A61K 9/4833; A61K 9/4866; B29C 43/006; B29L 2031/753

(57) ABSTRACT

The present invention provides an oral pharmaceutical composition of isotretinoin having enhanced bioavailability, wherein said composition is in the form of a solid dispersion comprising isotretinoin and a pharmaceutically acceptable matrix. The present invention further relates to a process for preparing the oral pharmaceutical composition of the present invention.

### FIELD OF THE INVENTION

The present invention provides an oral pharmaceutical composition of isotretinoin having enhanced bioavailability, wherein said composition is in the form of a solid dispersion comprising isotretinoin and a pharmaceutically acceptable matrix. The present invention further relates to a process for preparing the oral pharmaceutical composition of the present invention.

### BACKGROUND OF THE INVENTION

Isotretinoin is a retinoid (also known as 13-cis retinoic acid). Owing to its low water solubility, the oral bioavailability of isotretinoin is low. PCT Publication No. WO 00/25772 discloses that the presently marketed formulation of isotretinoin, i.e., Accutane®, contains isotretinoin at a mean particle size of about 100 μm resulting in only 20% oral bioavailability. Therefore, this application discloses a formulation of isotretinoin having a reduced particle size, thereby enhancing the oral bioavailability.

U.S. Pat. Nos. 7,435,427 and 8,367,102 cover the marketed formulation of Absorica®. These patents disclose capsules comprising a semi-solid suspension of isotretinoin containing at least two lipidic excipients, one having an HLB value equal to or greater than 10 and the other being an oily vehicle. These patents are based on the use of the “Lidose technology” to provide a formulation of isotretinoin with enhanced bioavailability.

The oral bioavailability of a drug is affected by various factors which include aqueous solubility, first pass effect, or food-effect. Out of these, the solubility of a drug in water is one of the major factors influencing the bioavailability of that drug. The bioavailability of a poorly soluble drug can be enhanced by various methods, which include particle size reduction, formation of salts or esters, complexation, or formation of solid dispersions. Isotretinoin is poorly soluble in water. Therefore, there is a need to develop a composition of isotretinoin having enhanced bioavailability. The present inventors have developed an oral pharmaceutical composition of isotretinoin having enhanced bioavailability, wherein said composition is in the form of a solid dispersion comprising isotretinoin and a pharmaceutically acceptable matrix.

### SUMMARY OF THE INVENTION

The present invention provides an oral pharmaceutical composition of isotretinoin having enhanced bioavailability, wherein said composition is in the form of a solid dispersion comprising isotretinoin and a pharmaceutically acceptable matrix. The present invention further provides a process for preparing the oral pharmaceutical composition of the present invention. It also provides a method of treating acne by administering the oral pharmaceutical composition of the present invention.

### DETAILED DESCRIPTION OF THE INVENTION

In one aspect, the present invention provides an oral pharmaceutical composition of isotretinoin having enhanced bioavailability, wherein said composition is in the form of a solid dispersion comprising isotretinoin and a pharmaceutically acceptable matrix.

In one embodiment of the above aspect, the pharmaceutically acceptable matrix is a polymeric matrix, a non-polymeric matrix, or a combination thereof.

The polymeric matrix includes, but is not limited to, hydroxypropylmethyl cellulose; hydroxypropyl cellulose; hydroxyethyl cellulose; hydroxymethyl cellulose; carboxymethyl cellulose; sodiumcarboxymethyl cellulose; carboxymethylcellulose calcium; polyvinyl pyrrolidone; polyethylene oxide; polyvinyl alcohol; methyl cellulose; ethyl cellulose; propyl cellulose; ethylmethyl cellulose; isopropyl cellulose; ethylpropyl cellulose; butyl cellulose; benzyl cellulose; cellulose esters such as cellulose acetate, cellulose butyrate, cellulose propionate, cellulose butyrate, and cellulose acetate propionate; cellulose cyanoalkyl ethers such as cyanoethyl cellulose, cyanomethyl cellulose, cyanoethylmethyl cellulose, and cyanopropyl cellulose; methacrylic acid-acrylic acid copolymers (e.g., Eudragit® RS, Eudragit® RL, Eudragit® NE, Eudragit® RS PO, and Eudragit® RL PO); methacrylic acid copolymers; hydroxypropyl methylcellulose phthalate; hydroxypropyl methylcellulose acetate succinate; cellulose acetate phthalate; and mixtures thereof.

The non-polymeric matrix includes, but is not limited to, sugar and sugar alcohols for example sucrose, lactose, fructose, maltose, raffinose, sorbitol, lactitol, mannitol, maltitol, erythritol, threitol, adonitol, arabitol, xylitol, dulcitol, inositol, trehalose, isomalt, inulin, and maltodextrin; cyclodextrin, for example β-cyclodextrin and hydroxypropyl-β-cyclodextrin; polyethylene glycol; polyethylene glycol esters; medium chain triglycerides; fatty acids; fatty alcohols; waxes; fatty acid esters; polyoxyethylene sorbitan fatty acid esters; urea; and mixtures thereof.

In another embodiment of the above aspect, the pharmaceutically acceptable matrix is present in an amount of about 1% w/w to about 90% w/w by total weight of the composition; preferably in an amount of about 50% w/w to 85% w/w by total weight of the composition.

In another embodiment of the above aspect, said composition comprises isotretinoin in an amount of about 1 mg to 100 mg, 5 mg to 50 mg, 10 mg to 40 mg, 9 mg to 36 mg, or 8 mg to 32 mg.

In another embodiment of the above aspect, said composition comprises isotretinoin in an amount of about 40 mg.

In another embodiment of the above aspect, said composition comprises isotretinoin in an amount of about 32 mg.

In another embodiment of the above aspect, said composition comprises isotretinoin in an amount of about 16 mg.

In another embodiment of the above aspect, said pharmaceutical composition is in the form of a solid dosage form comprising a solid dispersion of isotretinoin and a pharmaceutically acceptable matrix.

In another embodiment of the above aspect, said pharmaceutical composition further comprises one or more pharmaceutically acceptable excipients, for example, binders, disintegrants, fillers, lubricants, glidants, surfactants, stabilizing agents, colors, flavors, preservatives, and combinations thereof.

In yet another embodiment, said oral pharmaceutical composition is stable when stored at 40° C. and 75% relative humidity or at 25° C. and 60% relative humidity for a period of at least three months or to the extent necessary for the use of the composition.

In another aspect of the present invention, there is provided a process for preparing said solid dispersion, wherein the process is a solvent evaporation method, melting method, kneading method, co-grinding method, co-precipitation method, freeze-drying, coating, or adsorbing the drug onto carrier particles.

In one embodiment of the above aspect, said process comprises:
a) dissolving isotretinoin and a pharmaceutically acceptable matrix in a solvent; and
b) evaporating the solvent to form a solid dispersion of isotretinoin.

In another embodiment of the above aspect, said process comprises:
a) dissolving isotretinoin and one or more excipients in a solvent;
b) coating or adsorbing the solution of step a) onto a pharmaceutically acceptable matrix to form solid particles or granules; and
c) filing the solid particles or granules of step b) into capsules or compressing into tablets with one or more pharmaceutically acceptable excipients.

In another aspect, the present invention provides an oral pharmaceutical composition of isotretinoin having enhanced bioavailability, wherein said composition, when administered orally, provides equivalent efficacy at a lower dose of isotretinoin in comparison to the marketed Absorica® capsules, wherein said dose is at least 10% lower.

In another aspect, the present invention provides an oral pharmaceutical composition of isotretinoin having enhanced bioavailability, wherein said composition, when administered orally, provides equivalent efficacy at a lower dose of isotretinoin in comparison to the marketed Absorica® capsules, wherein said dose is at least 20% lower.

In another aspect, the present invention provides an oral pharmaceutical composition of isotretinoin having enhanced bioavailability wherein said composition exhibits reduced food effect as indicated by comparable Cₘₐₓ and AUC in fasting and fed states.

In still another aspect, the present invention provides a method of treating acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging by administering to the individual in need thereof the oral pharmaceutical composition of the present invention.

In one embodiment of the above aspect, the present invention provides a method of treating acne by administering to the individual in need thereof the oral pharmaceutical composition of the present invention.

The term “isotretinoin” refers to isotretinoin in its crystalline or amorphous form, its esters, salts, or derivatives thereof.

The term “solid dispersion” refers to a solidified form of a drug obtained by dispersing or dissolving the drug in a matrix. In the solid dispersion the drug is present in a molecular state, colloidal state, metastable state, or an amorphous state.

Various processes for preparing solid dispersions include solvent evaporation method, melting method, kneading method, co-grinding method, co-precipitation method, freeze-drying, coating, or adsorbing the drug onto carrier particles.

The term “solvent” as used herein refers to any solvent or solvent mixture, aqueous and non-aqueous solvents, protic or aprotic solvents; for example, water, alcohols, esters, halogenated hydrocarbons, ketones, ethers, and mixtures thereof. Examples of alcohols include, primary, secondary and tertiary alcohols, for example methanol, ethanol, n-propanol, isopropanol, and butanol. The esters may include one or more of ethyl acetate, n-propyl acetate, isopropyl acetate, and n-butyl acetate. Examples of halogenated hydrocarbons include dichloromethane, chloroform, and 1,2-dichloroethane. Examples of ketones include acetone, methyl ethyl ketone, and the like. Examples of ethers include diethyl ether and tetrahydrofuran.

The bioequivalence is established by comparing pharmacokinetic parameters for example, AUC and Cₘₐₓ of the pharmaceutical composition of the present invention with Absorica® capsules in healthy human subjects in fed as well as fasting conditions.

The term “AUC” refers to the area under the time/plasma concentration curve after administration of the pharmaceutical composition; AUC_{0-infinity} denotes the area under the plasma concentration versus time curve from time 0 to infinity; AUC₀₋ₜ denotes the area under the plasma concentration versus time curve from time 0 to time t.

The term “Cₘₐₓ” refers to the maximum concentration of isotretinoin in the blood following administration of the pharmaceutical composition.

The term “tₘₐₓ” refers to the time in hours when Cₘₐₓ is achieved following administration of the pharmaceutical composition.

The term “food effect” as used herein refers to food-drug interactions which either decrease or increase the extent of drug absorption. It refers to a relative difference in AUC, Cₘₐₓ and/or tₘₐₓ of a drug, when said drug or a formulation thereof is administered orally to a human, concomitantly with food or in a fed state as compared to when administered in a fasted state or without food.

The one or more additional pharmaceutically acceptable excipients are selected from binders, disintegrants, fillers, lubricants, glidants, surfactants, stabilizing agents, antioxidants, alkaline stabilizers, colors, flavors, preservatives, and combinations thereof.

Examples of binders include, but are not limited to, methyl cellulose, hydroxypropyl cellulose, hydroxypropylmethyl cellulose, polyvinyl pyrrolidone, poloxamer, gelatin, ethyl cellulose, polyvinyl alcohol, pullunan, pregelatinized starch, agar, tragacanth, sodium alginate, propylene glycol, gum arabic, and mixtures thereof.

Examples of disintegrants include, but are not limited to, cross-linked polyvinyl pyrrolidone, sodium starch glycolate, cross-linked sodiumcarboxymethyl cellulose, calcium carboxymethyl cellulose, alginic acid, alginates, pregelatinized starch, starch and its derivatives, low-substituted hydroxypropyl cellulose, and mixtures thereof.

Examples of fillers include, but are not limited to, starch, lactose, sucrose, glucose, sorbitol, calcium carbonate, calcium phosphate dibasic, calcium phosphate tribasic, calcium sulfate, microcrystalline cellulose, silicified microcrystalline cellulose, dextrates, dextrins, dextrose, fructose, lactitol, mannitol, sorbitol, pregelatinized starch, and mixtures thereof.

Examples of lubricants and glidants include, but are not limited to, colloidal anhydrous silica, magnesium stearate, calcium stearate, stearic acid, talc, hydrogenated castor oil, sucrose esters of fatty acid, and mixtures thereof.

Examples of antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof.

Examples of alkaline stabilizers include, but are not limited to, sodium hydroxide, potassium hydroxide, sodium carbonate or bicarbonate, potassium carbonate or bicarbonate, lithium hydroxide, triethylamine, meglumine, methylamine, and mixtures thereof.

Examples of suitable preservatives include, but are not limited to, methyl paraben, ethyl paraben, propyl paraben, butyl paraben, benzoic acid, sodium benzoate, benzyl alcohol, sorbic acid, potassium sorbate, and mixtures thereof.

The term “stable” as used herein, refers to chemical stability, wherein not more than 1.5% w/w of total related substances are formed on storage at accelerated conditions of stability at 40° C. and 75% relative humidity or at 25° C. and 60% relative humidity for a period of at least three months or to the extent necessary for use of the composition.

The invention may be further illustrated by the following examples, which are for illustrative purposes only and should not be construed as limiting the scope of the invention in anyway.

### EXAMPLES

### Example 1

| Ingredient | Quantity (% w/w) |
|---|---|
| Isotretinoin | 11.08 |
| Lactose anhydrous | 66.48 |
| Butylated hydroxy anisole | 0.28 |
| Povidone K30 | 5.54 |
| Polyoxyl 40 hydrogenated castor | 16.62 |
| oil (Kolliphor ® RH 40) | |

### Procedure:

1. Povidone K30 and Kolliphor® RH 40 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 2

| Ingredient | Quantity (% w/w) |
|---|---|
| Isotretinoin | 11.08 |
| Lactose anhydrous | 66.48 |
| Butylated hydroxy anisole | 0.28 |
| Povidone K30 | 5.54 |
| Stearoyl polyoxyl-32 glycerides | 16.62 |
| (Gelucire ® 50/13) | |

### Procedure:

1. Povidone K30 and Gelucire® 50/13 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 3

| Ingredient | Quantity (% w/w) |
|---|---|
| Isotretinoin | 10.23 |
| Lactose anhydrous | 61.37 |
| Butylated hydroxy anisole | 0.26 |
| Povidone K30 | 5.12 |
| Polyoxyl 40 hydrogenated castor | 11.51 |
| oil (Kolliphor ® RH 40) | |
| Stearoyl polyoxyl-32 glycerides | 11.51 |
| (Gelucire ® 50/13) | |

### Procedure:

1. Povidone K30, Kolliphor® RH 40 and Gelucire® 50/13 were dissolved in a mixture of dichloromethane and ethanol (in a ratio of 68.42:31.58) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 4

| Ingredient | Quantity (% w/w) |
|---|---|
| Isotretinoin | 10.23 |
| Lactose anhydrous | 61.37 |
| Butylated hydroxy anisole | 0.26 |
| Povidone K30 | 5.12 |
| Polyoxyl 40 hydrogenated castor | 7.67 |
| oil (Kolliphor ® RH 40) | |
| Stearoyl polyoxyl-32 glycerides | 15.35 |
| (Gelucire ® 50/13) | |

### Procedure:

1. Povidone K30, Kolliphor® RH 40 and Gelucire® 50/13 were dissolved in a mixture of dichloromethane and ethanol (in a ratio of 68.42:31.58) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 5

| Ingredient | Quantity (% w/w) |
|---|---|
| Isotretinoin | 9.28 |
| Lactose anhydrous | 74.25 |
| Croscarmellose sodium | 4.64 |
| Butylated hydroxy anisole | 0.23 |
| Povidone K30 | 4.64 |
| Polaxomer 188 | 6.96 |

### Procedure:

1. Povidone K30 and polaxomer 188 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 6

| Ingredient | Quantity (% w/w) |
|---|---|
| Isotretinoin | 8.67 |
| Lactose anhydrous | 69.41 |
| Croscarmellose sodium | 4.34 |
| Butylated hydroxy anisole | 0.22 |
| Povidone K30 | 4.34 |
| Stearoyl polyoxyl-32 glycerides | 13.02 |
| (Gelucire ® 50/13) | |

### Procedure:

1. Povidone K30 and Gelucire® 50/13 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 7

| Ingredient | Quantity (% w/w) |
|---|---|
| Isotretinoin | 8.67 |
| Lactose anhydrous | 69.41 |
| Croscarmellose sodium | 4.34 |
| Butylated hydroxy anisole | 0.22 |
| Povidone K30 | 4.34 |
| Polyoxyl 40 hydrogenated castor oil | 13.02 |
| (Kolliphor ® RH 40) | |

### Procedure:

1. Povidone K30 and Kolliphor® RH 40 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 8

| Ingredient | Quantity (% w/w) |
|---|---|
| Isotretinoin | 9.07 |
| Lactose anhydrous | 72.55 |
| Croscarmellose sodium | 4.54 |
| Butylated hydroxy anisole | 0.23 |
| Hydroxypropylmethyl cellulose E3 LV | 9.07 |
| Polaxomer 188 | 4.54 |

### Procedure:

1. Hydroxypropylmethyl cellulose E3 LV and polaxomer 188 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 9

| Ingredient | Quantity (% w/w) |
|---|---|
| Isotretinoin | 9.17 |
| Lactose anhydrous | 73.39 |
| Croscarmellose sodium | 4.59 |
| Butylated hydroxy anisole | 0.23 |
| Hydroxypropylmethyl cellulose E15 LV | 3.45 |
| Stearoyl polyoxyl-32 glycerides (Gelucire ® 50/13) | 9.17 |

### Procedure:

1. Hydroxypropylmethyl cellulose E15 LV and Gelucire® 50/13 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 10

| Ingredient | Quantity (% w/w) |
|---|---|
| Isotretinoin | 8.87 |
| Lactose anhydrous | 70.96 |
| Croscarmellose sodium | 4.43 |
| Butylated hydroxy anisole | 0.22 |
| Polyethylene glycol 6000 | 8.87 |
| Polaxomer 188 | 6.65 |

### Procedure:

1. Polyethylene glycol 6000 and poloxamer 188 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 11

| Ingredient | Quantity (% w/w) |
|---|---|
| Isotretinoin | 8.68 |
| Lactose anhydrous | 69.40 |
| Croscarmellose sodium | 4.34 |
| Butylated hydroxy anisole | 0.22 |
| Polyethylene glycol 6000 | 8.68 |
| Docusate sodium | 8.68 |

### Procedure:

1. Polyethylene glycol 6000 and docusate sodium were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 12

| Ingredient | Quantity (% w/w) |
|---|---|
| Isotretinoin | 8.32 |
| Lactose anhydrous | 66.53 |
| Croscarmellose sodium | 4.16 |
| Butylated hydroxy anisole | 0.21 |
| Polyethylene glycol 6000 | 8.32 |
| Stearoyl polyoxyl-32 glycerides | 12.46 |
| (Gelucire ® 50/13) | |

### Procedure:

1. Polyethylene glycol 6000 and Gelucire® 50/13 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 13

| Ingredient | Quantity (% w/w) |
|---|---|
| Isotretinoin | 8.32 |
| Lactose anhydrous | 66.53 |
| Croscarmellose sodium | 4.16 |
| Butylated hydroxy anisole | 0.21 |
| Polyethylene glycol 6000 | 8.32 |
| Polyoxyl 40 hydrogenated castor oil | 12.46 |
| (Kolliphor ® RH 40) | |

### Procedure:

1. Polyethylene glycol 6000 and Kolliphor® RH 40 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous and croscarmellose sodium were loaded into a fluidized bed processor bowl for granulation.
4. The solution of step 2 was sprayed over the loaded lactose and croscarmellose sodium of step 3 to form granules.
5. The granules of step 4 were dried and filled into capsules.

### Example 14

| Ingredients | Quantity (% w/w) |
|---|---|
| Isotretinoin | 11.73 |
| Lactose anhydrous | 70.38 |
| Butylated hydroxy anisole | 0.29 |
| Stearic acid | 11.73 |
| Polyoxyl 40 hydrogenated castor oil | 5.87 |
| (Kolliphor ® RH 40) | |

### Procedure:

1. Stearic acid and Kolliphor® RH 40 were dissolved in a mixture of dichloromethane and ethanol (in a ratio of 70.37:29.63) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 15

| Ingredients | Quantity (% w/w) |
|---|---|
| Isotretinoin | 11.73 |
| Lactose anhydrous | 70.38 |
| Butylated hydroxy anisole | 0.29 |
| Hard fat (Gelucire ® 43/01) | 11.73 |
| Polyoxyl 40 hydrogenated castor oil | 5.87 |
| (Kolliphor ® RH 40) | |

### Procedure:

1. Gelucire® 43/01 and Kolliphor® RH 40 were dissolved in a mixture of dichloromethane and ethanol (in a ratio of 81.48:18.52) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 16

| Ingredients | Quantity (% w/w) |
|---|---|
| Isotretinoin | 11.73 |
| Lactose anhydrous | 70.38 |
| Butylated hydroxy anisole | 0.29 |
| Glyceryl behenate (Compritol ® 888) | 11.73 |
| Polyoxyl 40 hydrogenated castor oil | 5.87 |
| (Kolliphor ® RH 40) | |

### Procedure:

1. Compritol® 888 and Kolliphor® RH 40 were dissolved in a mixture of dichloromethane and ethanol (in a ratio of 66.67:33.33) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Example 17

| Ingredients | Quantity (% w/w) |
|---|---|
| Isotretinoin | 11.73 |
| Lactose anhydrous | 70.38 |
| Butylated hydroxy anisole | 0.29 |
| Polyoxyl 40 hydrogenated castor oil | 17.60 |
| (Kolliphor ® RH 40) | |

### Procedure:

1. Kolliphor® RH 40 was dissolved in a mixture of dichloromethane and ethanol (in a ratio of 76:24) under stirring to form a clear solution.
2. Butylated hydroxy anisole and isotretinoin were dissolved in the solution of step 1 under stirring to form a clear solution.
3. Lactose anhydrous was loaded into a fluidized bed processor bowl for granulation.
4. The solution of step 2 was sprayed over the loaded lactose of step 3 to form granules.
5. The granules of step 4 were dried and filled into capsules.

### Dissolution Studies

The release profile of the pharmaceutical compositions of Examples 1-17, is as given below:

| | |
|---|---|
| Dissolution Media | pH 10.5 Borate buffer |
| Apparatus/RPM/Vol | USP Type I/100/900 mL |

| Time | | | | | Exam- | Exam- |
|---|---|---|---|---|---|---|
| (Min) | Example 1 | Example 2 | Example 3 | Example 4 | ple 5 | ple 6 |
| 15 | 70 | 2 | 79 | 49 | 74 | 70 |
| 30 | 84 | 57 | 86 | 86 | 91 | 89 |
| 45 | 88 | 87 | 86 | 91 | 92 | 89 |
| 60 | 92 | 97 | 84 | 90 | 88 | 78 |
| Time | | | | Example | Exam- | Exam- |
| (Min) | Example 7 | Example 8 | Example 9 | 10 | ple 11 | ple 12 |
| 15 | 88 | 67 | 64 | 59 | 4 | 20 |
| 30 | 97 | 79 | 86 | 79 | 17 | 60 |
| 45 | 90 | 90 | 94 | 81 | 32 | 69 |
| 60 | 96 | 91 | 90 | 83 | 38 | 70 |

| Time | Example | Example | Example | Example | Example |
|---|---|---|---|---|---|
| (Min) | 13 | 14 | 15 | 16 | 17 |
| 15 | 34 | 79 | 59 | 72 | 77 |
| 30 | 68 | 103 | 72 | 90 | 88 |
| 45 | 51 | 104 | 81 | 86 | 91 |
| 60 | 73 | 102 | 82 | 83 | 90 |

1. An oral pharmaceutical composition of isotretinoin having enhanced bioavailability wherein said composition is in the form of a solid dispersion comprising isotretinoin and a pharmaceutically acceptable matrix.

2. The oral pharmaceutical composition according to claim 1, wherein the pharmaceutically acceptable matrix is a polymeric matrix, a non-polymeric matrix, or a combination thereof.

3. The oral pharmaceutical composition according to claim 2, wherein the polymeric matrix is selected from the group consisting of hydroxypropylmethyl cellulose, hydroxypropyl cellulose, hydroxyethyl cellulose, hydroxymethyl cellulose, carboxymethyl cellulose, sodium carboxymethyl cellulose, carboxymethyl cellulose calcium, polyvinylpyrrolidone, polyethylene oxide, polyvinyl alcohol, methyl cellulose, ethyl cellulose, propyl cellulose, ethylmethyl cellulose, isopropyl cellulose, ethylpropyl cellulose, butyl cellulose, benzyl cellulose, cellulose esters, cellulose cyanoalkyl ethers, methacrylic acid-acrylic acid copolymers, methacrylic acid copolymers, hydroxypropyl methylcellulose phthalate, hydroxypropyl methylcellulose acetate succinate, cellulose acetate phthalate, and mixtures thereof.

4. The oral pharmaceutical composition according to claim 2, wherein the non-polymeric matrix is selected from the group consisting of sugars, sugar alcohols, cyclodextrin, polyethylene glycol, polyethylene glycol esters, medium chain triglycerides, fatty acids, fatty alcohols, waxes, fatty acid esters, polyoxyethylene sorbitan fatty acid esters, urea, and mixtures thereof.

5. The oral pharmaceutical composition according to claim 1, wherein the pharmaceutically acceptable matrix is present in an amount of about 1% w/w to about 90% w/w by total weight of the composition.

6. The oral pharmaceutical composition according to claim 5, wherein the pharmaceutically acceptable matrix is present in an amount of about 50% w/w to about 85% w/w by total weight of the composition.

7. The oral pharmaceutical composition according to claim 1, wherein said composition comprises isotretinoin in an amount of about 1 mg to 100 mg, 5 mg to 50 mg, 10 mg to 40 mg, 9 mg to 36 mg, or 8 mg to 32 mg.

8. The oral pharmaceutical composition according to claim 7, wherein said composition comprises isotretinoin in an amount of about 40 mg.

9. The oral pharmaceutical composition according to claim 7, wherein said composition comprises isotretinoin in an amount of about 32 mg, 28 mg, 24 mg, 20 mg, 16 mg, or 8 mg.

10. (canceled)

11. The oral pharmaceutical composition according to claim 1, wherein said composition is in the form of a solid dosage form comprising a solid dispersion of isotretinoin and a pharmaceutically acceptable matrix.

12. The oral pharmaceutical composition according to claim 1, wherein said composition further comprises one or more pharmaceutically acceptable excipients selected from the group comprising binders, disintegrants, fillers, lubricants, glidants, surfactants, stabilizing agents, colors, flavors, preservatives, and combinations thereof.

13. The oral pharmaceutical composition according to claim 1, wherein said composition is stable when stored at 40° C. and 75% relative humidity or at 25° C. and 60% relative humidity for a period of at least three months.

14. A process for preparing the oral pharmaceutical composition of claim 1, wherein said process is solvent evaporation method, melting method, kneading method, co-grinding method, co-precipitation method, freeze-drying, coating, or adsorbing the drug onto carrier particles.

15. The process according to claim 14, wherein said process comprises:
a) dissolving isotretinoin and a pharmaceutically acceptable matrix in a solvent; and
b) evaporating the solvent to form a solid dispersion of isotretinoin.

16. The process according to claim 14, wherein said process comprises:
a) dissolving isotretinoin and one or more excipients in a solvent;
b) coating or adsorbing the solution of step a) onto a pharmaceutically acceptable matrix to form solid particles or granules; and
c) filling the solid particles or granules of step b) into capsules or compressing into tablets with one or more pharmaceutically acceptable excipients.

17. The oral pharmaceutical composition according to claim 1, wherein said composition, when administered orally, provides equivalent efficacy at a lower dose of isotretinoin in comparison to the marketed Absorica® capsules, wherein said dose is at least 10% lower.

18. The oral pharmaceutical composition according to claim 1, wherein said composition, when administered orally, provides equivalent efficacy at a lower dose of isotretinoin in comparison to the marketed Absorica® capsules, wherein said dose is at least 20% lower.

19. The oral pharmaceutical composition according to claim 1, wherein said composition exhibits reduced food effect as indicated by comparable Cₘₐₓ and AUC in fasting and fed states.

20. The oral pharmaceutical composition according to claim 1, wherein said composition is used for the treatment of acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging.

21. The oral pharmaceutical composition according to claim 20, wherein said composition is used for the treatment of acne.

22. A method of treating acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging comprising administering to a patient in a need thereof a therapeutically effective amount of the oral pharmaceutical composition of claim 1.

23. The method according to claim 22, wherein the patient has acne.

24. The oral composition according to claim 1, wherein said composition releases more than 50% of isotretinoin in 15 minutes in a media with a pH of 10.5.
